(19) United States
(12) Patent Application Publication
(10) Pub. No.: US 2015/0258300 A1
(43) Pub. Date: Sep. 17, 2015

(54) HUMIDIFIER AND BREATHING APPARATUS USING THE SAME

(71) Applicant: LEAD DATA INC., Hsinchu County (TW)

(72) Inventors: SHU-HUNG LIN, Hsinchu County (TW); CHIH-WEI FENG, Taipei City (TW); CHIH-YANG HSIAO, Hsinchu County (TW)

(21) Appl. No.: 14/620,511

(22) Filed: Feb. 12, 2015

(30) Foreign Application Priority Data
Mar. 14, 2014 (TW) 103204419

(51) Int. Cl. A61M 16/16; A61M 16/08; A61M 16/10; A61M 16/06

(52) U.S. Cl. CPC A61M 16/16; A61M 16/06; A61M 16/0816; A61M 16/109

(57) ABSTRACT

A humidifier including a water container, a dividing plate and an airway structure is provided. The water container has a top and a bottom opposite to the top. The top of the water container has a first air outlet. The dividing plate is disposed in the water container and dividing an internal space of the water container into a first space and a second space. The first space is defined between the bottom and the dividing plate and the second space is defined between the dividing plate and the top. The airway structure is disposed in the second space and having a certain distance to the top. The airway structure has an air inlet and a second air outlet. The air inlet of the airway structure is connected to a sidewall of the water container and the second air outlet is connected to the dividing plate.

### FIELD OF THE INVENTION

The present invention relates to a humidifier, and more particularly to a humidifier and a breathing apparatus using the same.

### BACKGROUND OF THE INVENTION

Continuous positive airway pressure (CPAP) breathing apparatus is a physical therapy for the patients having sleep apnea. Basically, the continuous positive airway pressure breathing apparatus is equipped with a humidifier which is for increasing the humidity of the supplied air, so that the patent can feel more comfortable while breathing.

In a conventional breathing apparatus, the humidifier includes a water container. The water container has a cover body and a sidewall. The sidewall has an air inlet and the cover body has an air outlet. In the air humidification process, the air provided by an air supply device of the breathing apparatus first flows into the water container through the air inlet, is humidified in the water container and then flows out through the air outlet. However, a conventional breathing apparatus equipped with the humidifier may make a noise louder than a breathing apparatus without the humidifier and the louder noise may affect the sleep quality of a user. Therefore, it is important to design a breathing apparatus having reduced noise.

### SUMMARY OF THE INVENTION

For making the above and other purposes, features and benefits become more readily apparent to those ordinarily skilled in the art, the preferred embodiments and the detailed descriptions with accompanying drawings will be put forward in the following descriptions.

One object of the present invention is to provide a humidifier able to reduce the noise generated during the air humidification process.

Another object of the present invention is to provide a breathing apparatus able to reduce the noise while being operated.

The present invention provides a humidifier, which includes a water container, a dividing plate and an airway structure. The water container has a top and a bottom opposite to the top. The top of the water container has a first air outlet. The dividing plate is disposed in the water container and dividing an internal space of the water container into a first space and a second space. The first space is defined between the bottom of the water container and the dividing plate and the second space is defined between the dividing plate and the top of the water container. The airway structure is disposed in the second space and having a certain distance to the top of the water container. The airway structure has an air inlet and a second air outlet. The air inlet of the airway structure is connected to a sidewall of the water container and the second air outlet of the airway structure is connected to the dividing plate.

In one embodiment, the airway structure includes a fence and a cover plate. The fence is disposed on the dividing plate. The cover plate is disposed above the fence and having a certain distance to the top of the water container. An airway space is formed by the cover plate, the fence and the dividing plate.

In one embodiment, the aforementioned humidifier further includes a connection structure connected between the cover plate and the top of the water container.

In one embodiment, the connection structure and the cover plate are integrated to have a one-piece structure.

In one embodiment, the fence and the dividing plate are integrated to have a one-piece structure.

In one embodiment, the airway structure is an airway pipe.

In one embodiment, there is a certain distance between the dividing plate and the sidewall of the water container.

In one embodiment, the dividing plate has at least one through hole.

In one embodiment, the top of the water container further has a dividing portion extending toward the internal of the water container.

In one embodiment, the air inlet is formed close to the top of the water container.

In one embodiment, the water container includes a box body, cover body and a metal bottom plate. The box body has a top opening and a bottom opening opposite to the top opening. The cover body is disposed on the top opening to form the top of the water container. The metal bottom plate is disposed on the bottom opening to form the bottom of the water container.

The present invention further provides a breathing apparatus, which includes a humidifier, a heating device and an air supply device. The humidifier includes a water container, a dividing plate and an airway structure. The water container has a top and a bottom opposite to the top. The top of the water container has a first air outlet. The dividing plate is disposed in the water container and dividing an internal space of the water container into a first space and a second space. The first space is defined between the bottom of the water container and the dividing plate and the second space is defined between the dividing plate and the top of the water container. The airway structure is disposed in the second space and having a certain distance to the top of the water container. The airway structure has an air inlet and a second air outlet. The air inlet of the airway structure is connected to a sidewall of the water container and the second air outlet of the airway structure is connected to the dividing plate. The heating device is for supporting the humidifier and heating the bottom of the water container. The air supply device is for providing an air. The air flows into the humidifier through the air inlet of the humidifier.

In summary, because the airway structure is not in contact with the top of the water container and the internal space of the water container is divided into a plurality of relatively small spaces by the dividing plate, the noise generated during the air humidification process of the humidifier of the present invention is reduced. Accordingly, the breathing apparatus equipped with the humidifier of the present invention has reduced noises and consequentially a user can have good sleep quality.

### BRIEF DESCRIPTION OF THE DRAWINGS

The invention will become more readily apparent to those ordinarily skilled in the art after reviewing the following detailed description and accompanying drawings, in which:

FIG. 1 is a schematic three-dimensional view of a breathing apparatus in accordance with an embodiment of the present invention;

FIG. 2 is a schematic cross-sectional view of a humidifier of the breathing apparatus of FIG. 1 in accordance with an embodiment of the present invention;

FIG. 3 is a schematic three-dimensional exploded structural view of a humidifier in accordance with another embodiment of the present invention;

FIG. 4 is a schematic three-dimensional structural view of the dividing plate and the airway structure in FIG. 3 after being assembled;

FIG. 5 is a schematic three-dimensional structural view of the dividing plate, the airway structure and the cover body in FIG. 3 after being assembled; and

FIG. 6 is a schematic structural view of the cover body and the airway structure in FIG. 3 after being assembled.

### DETAILED DESCRIPTION OF PREFERRED EMBODIMENTS

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “front”, “back”, etc., is used with reference to the orientation of the Figure(s) being described. The components of the invention can be positioned in a number of different orientations. As such, the directional terminology is used for purposes of illustration and is in no way limiting. On the other hand, the drawings are only schematic and the sizes of components may be exaggerated for clarity. It is to be understood that other embodiments may be utilized and structural changes may be made without departing from the scope of the invention. Also, it is to be understood that the phraseology and terminology used herein are for the purpose of description and should not be regarded as limiting. The use of “including”, “comprising”, or “having” and variations thereof herein is meant to encompass the items listed thereafter and equivalents thereof as well as additional items. Accordingly, the drawings and descriptions will be regarded as illustrative in nature and not as restrictive.

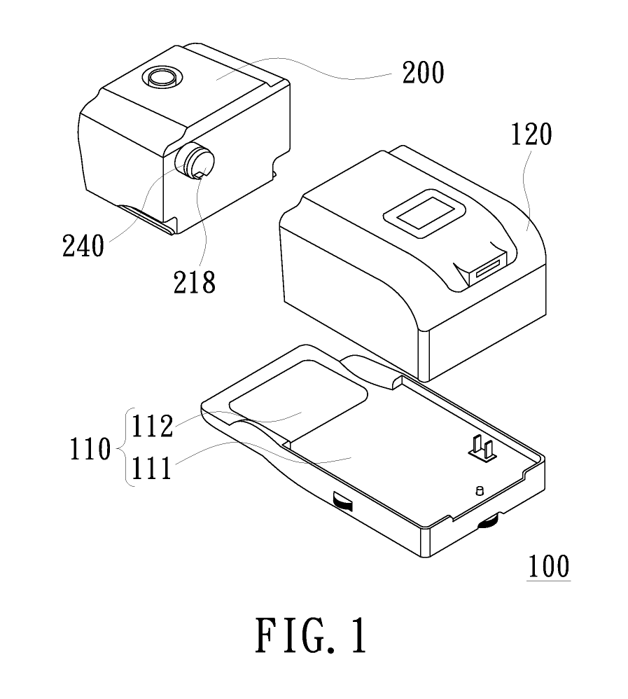

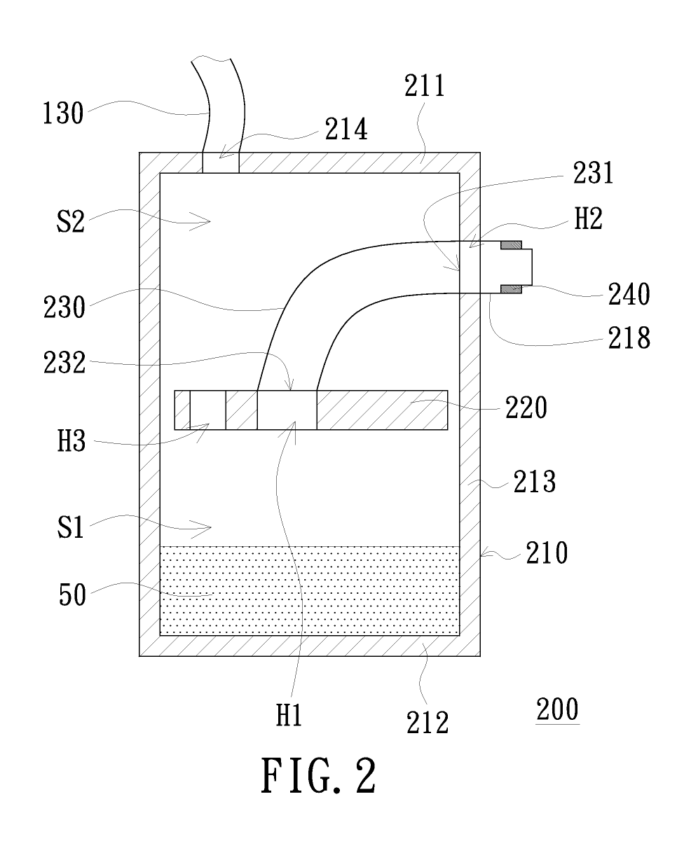

FIG. 1 is a schematic three-dimensional view of a breathing apparatus in accordance with an embodiment of the present invention. FIG. 2 is a schematic cross-sectional view of a humidifier of the breathing apparatus of FIG. 1 in accordance with an embodiment of the present invention. Please refer to FIGS. 1 and 2. The breathing apparatus 100 in the present embodiment may be a continuous positive airway pressure (CPAP) breathing apparatus; however, the present invention is not limited thereto. The breathing apparatus 100 includes a heating device 110, an air supply device 120 and a humidifier 200. The humidifier 200 includes a water container 210, a dividing plate 220 and an airway structure 230. The water container 210 has a top 211 and a bottom 212 opposite to the top 211. The top 211 of the water container 210 has a first air outlet 214. The dividing plate 220 is disposed in the water container 210 to divide the internal space of the water container 210 into a first space S1 and a second space S2. Specifically, the first space S1 is defined between the bottom 212 and the dividing plate 220 and the second space S2 is defined between the dividing plate 220 and the top 211. The airway structure 230 is disposed in the second space S2 and has a certain distance to the top 211; in other words, there is a certain distance between the airway structure 230 and the top 211 of the water container 210. The airway structure 230 has an air inlet 231 and a second air outlet 232 respectively at the two terminal ends thereof The air inlet 231 is connected to a sidewall 213 of the water container 230 and the second air outlet 232 is connected to the dividing plate 220. The heating device 110 is used to support the humidifier 200 and heat the bottom 212 of the water container 210; and consequentially, the liquid 50 (e.g., water) contained in the water container 210 is heated through the heated bottom 212. The air supply device 120 is for supplying air (not shown) to the humidifier 200 through the air inlet 231 of the humidifier 200. The heating device 110 in this embodiment includes a support base 111 and a heating element 112. The support base 111 is for supporting the water container 210 and the heating element 112, disposed on the support base 111, is for heating the bottom 212 of the water container 210. In addition, the support base 111 can be also used to support the air supply device 120.

The airway structure 230 in this embodiment may be an airway pipe. The pipe wall of the airway structure 230 has a certain distance to the top 211 of the water container 210; in other words, there is a distance between the pipe wall of the airway structure 230 and the top 211 of the water container 210 and the pipe wall of the airway structure 230 is not in contact with the top 211 of the water container 210. The second air outlet 232 of the airway structure 230 is, for instance, in communication with a through hole H1 of the dividing plate 220. The air inlet 231 of the airway structure 230 is, for instance, disposed close to the top 211 of the water container 210 and in communication with a through hole H2 of the sidewall 213. The sidewall 213 further has, for instance, a connection pipe 218 extending outwardly to connect to an air outlet (not shown) of the air supply device 120. One terminal end of the connection tube 218 is provided with, for instance, an annular sealing element 240 for the air leak proof Specifically, the annular sealing element 240 is used to prevent the air, provided by the air supply device 120, from being leaked from a connection part of the connection pipe 218 and the air outlet of the air supply device 120. In this embodiment, the annular sealing element 240 is made of silicon. In another embodiment, the airway pipe may extend out of the sidewall 213 from the through hole H1 and is directly connected to the air outlet (not shown) of the air supply device 120; and correspondingly, the aforementioned connection pipe 218 can be omitted.

In the breathing apparatus 100, the air provided by the air supply device 120 sequentially flows through the connection pipe 218, the through hole H2 of the sidewall 213, the air inlet 231 of the airway structure 230, the internal of the airway structure 230, the second air outlet 232 of the airway structure 230, the through hole H1 of the dividing plate 220 and eventually flows into the first space S1. Because the bottom 212 of the water container 210 is heated by the heating device 110 and consequentially the liquid 50 is heated by the heated bottom 212 of the water container 210, the first space S1 is fulfilled with moisture and the air in the first space S1 is humidified. The humidified air then flows upwardly from the first space S1. In this embodiment, the dividing plate 220 may have at least one through hole H3, through which the humidified air can flow upwardly into the second space S2. The humidified air then flows out of the water container 210 through the first air outlet 214 of the top 211 of the water container 210. Moreover, a gap may be formed between the dividing plate 220 and the sidewall 213, so that the humidified air can flow upwardly into the second space S2 through the gap and then flow out of the water container 210 through the first air outlet 214 of the top 211 of the water container 210. Furthermore, the breathing apparatus 100 in the present embodiment may further include a mask channel 130. Specifically, one terminal end of the mask channel 130 is connected to the first air outlet 214 and the other terminal end of the mask channel 130 is connected to a user; thus, the air flowing out of the first air outlet 214 is directed into a nasal cavity of the user.

In this embodiment, it is to be noted that there is a certain distance between the airway structure 230 and the top 211 of the water container 210, the pipe wall of the airway structure 230 is not in contact with the top 211 of the water container 210, and a plurality of relatively small spaces (such as the first and second spaces S1, S2) are formed in the internal of the water container 210 by the dividing plate 220. Thus, the noise generated when the air flowing through the humidifier 200 is reduced and accordingly the breathing apparatus 100 equipped with the humidifier 200 has reduced noise. As a result, a user can have good sleep quality without being disturbed by loud noise. Furthermore, it is to be noted that the airway structure 230 may be realized by a flexible airway pipe; thus, the air path can have flexible design.

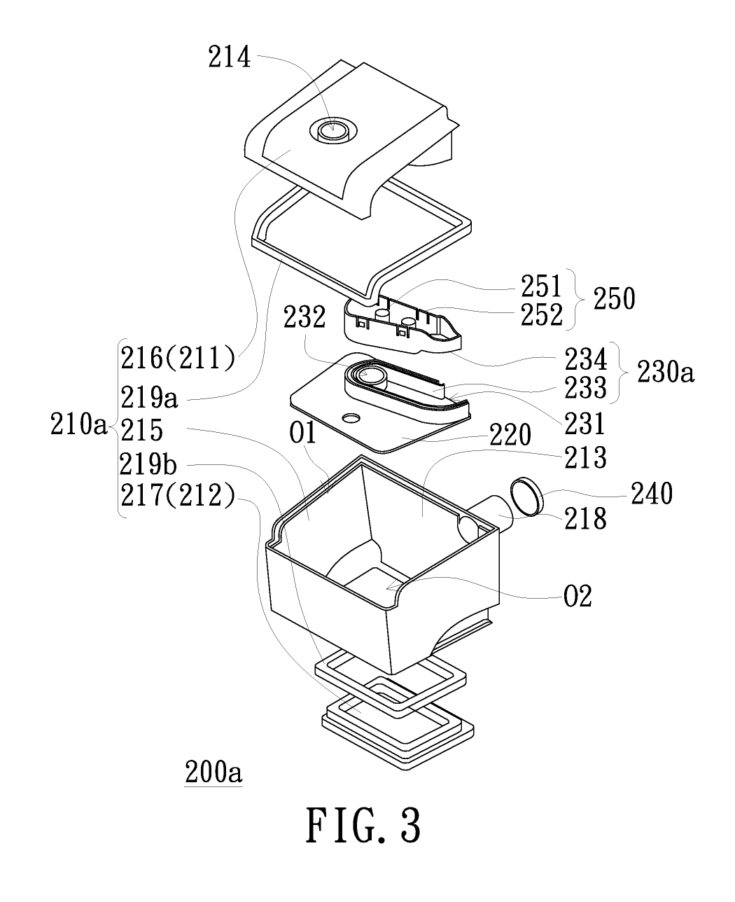

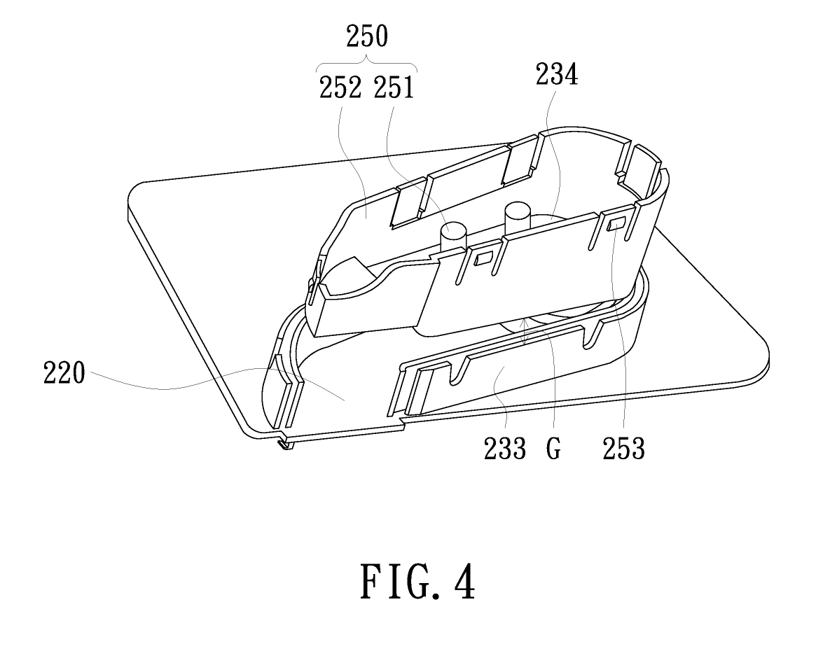

FIG. 3 is a schematic three-dimensional exploded structural view of a humidifier in accordance with another embodiment of the present invention. FIG. 4 is a schematic three-dimensional structural view of the dividing plate and the airway structure in FIG. 3 after being assembled. Please refer to FIGS. 3 and 4. The humidifier 200a in this embodiment has structure and advantages similar to that of the aforementioned humidifier 200, thus, only the differences between the two humidifiers 200 and 200a will be described as follow. The airway structure 230a of the humidifier 200a in this embodiment includes a fence 233 and a cover plate 234. The fence 233 is disposed on the dividing plate 220 and the cover plate 234 is disposed above the fence 233. The space corporately formed by the fence 233, the cover plate 234 and the dividing plate 220 is the airway space. The cover plate 234 in this embodiment has, for instance, a sheet form. When the components of the humidifier 200a are assembled, there is a certain distance between the cover plate 234 and the top 211 of the water container 210a so that the airway structure 230a is not in contact with the top 211 of the water container 210a. In this embodiment, the fence 233 and the dividing plate 220 may be integrated to have a one-piece structure. In another embodiment, the fence 233 and the dividing plate 220 are manufactured independently and the fence 233 is then fixed to the dividing plate 220.

The humidifier 200a in this embodiment may include a connection structure 250. The connection structure 250, connected between the cover plate 234 and the top 211 of the water container 210a, is for fixing the airway structure 230a and the dividing plate 220 in the water container 210a. In this embodiment, the connection structure 250 and the cover plate 234 may be integrated to have a one-piece structure. In another embodiment, the connection structure 250 and the dividing plate 234 may be manufactured independently and the connection structure 250 is then fixed to the cover plate 234. The connection structure 250 in this embodiment includes, for instance, a column body 251 and a sidewall 252. The column body 251 extends toward the top 211 of the water container 210a from the cover plate 234. The sidewall 252 extends toward the top 211 of the water container 210a from the edge of the cover plate 234. The column body 251 may be coupled to the top 211 of the water container 210a by means of clamp or lock, and the present invention is not limited thereto. The sidewall 252 may be provided with a connection structure, such as a clamp structure 253, for being connected with the top 211 of the water container 210a.

In another embodiment, the dividing plate 220 is fixed by being directly connected to the sidewall 214 of the water container 210a and the airway structure 230a is supported by the dividing plate 220; thus, the aforementioned connection structure 250 can be omitted. It is to be noted that in the embodiment of connecting the dividing plate 220 to the sidewall 214 of the water container 210a, the dividing plate 220 is provided with a plurality of through holes, through which the air can flow into the second space between the dividing plate 220 and the top 211 of the water container 210a.

The water container 210a in this embodiment includes a box body 215, a cover body 216 and a metal bottom plate 217. The box body 215 has a top opening O1 and a bottom opening O2 opposite to the top opening O1. The cover body 216 is disposed on the top opening O1 to form the top 211 of the water container 210a. The metal bottom plate 217 is disposed on the bottom opening O2 to form the bottom 212 of the water container 210a. The metal bottom plate 217 may be made of aluminum or other metal material with higher thermal conductivity so that the heat provided by the heating device of the breathing apparatus can be transmitted more efficiently. In addition, the sidewall 213 of the box body 215 may have a connection pipe 218 corresponding to the air inlet 231 of the airway structure 230a and connected to the air outlet of the air supply device 120 such as shown in FIG. 1. An annular sealing element 240 may be disposed at one terminal end of the connection pipe 218 to prevent the air provided by the air supply device 120 being leaked from the connection part between the connection pipe 218 and the air outlet of the air supply device 120.

The water container 210a in this embodiment may further include sealing elements 219a and 219b. The sealing element 219a is disposed between the box body 215 and the cover body 216. The sealing element 219b is disposed between the box body 215 and the metal bottom plate 217. By using the sealing elements 219a and 219b, the liquid in the water container 210a is prevented from being leaked from the connection parts between the box body 215 and the cover body 216 as well as the box body 215 and the metal bottom plate 217. The sealing elements 219a, 219b are, for instance, made of silicon; however, the present invention is not limited thereto.

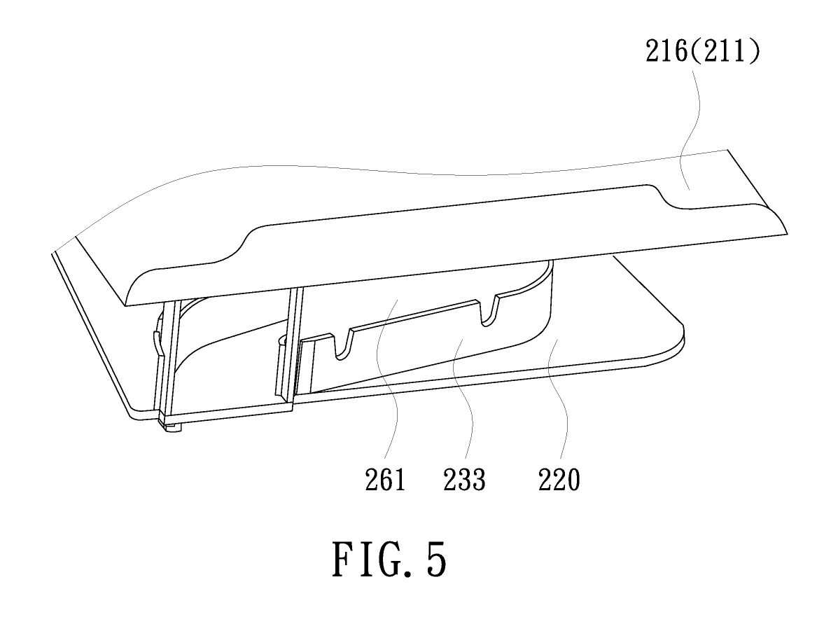

FIG. 5 is a schematic three-dimensional structural view of the dividing plate, the airway structure and the cover body in FIG. 3 after being assembled. Please refer to FIGS. 4 and 5. After the cover plate 234 and the fence 233 are assembled to each other, a gap G exists between the cover plate 234 and the fence 233. In addition, to prevent the air from being leaked through the gap G, the cover body 216 of the water container 210a may be designed to have an extending portion 261 extending toward the fence 233. The extending portion 261 is coupled to the fence 233 and covers the gap G so as to void the air leak. In addition, the clamp structure 253 of the sidewall 252 may be clamped to the extending portion 261.

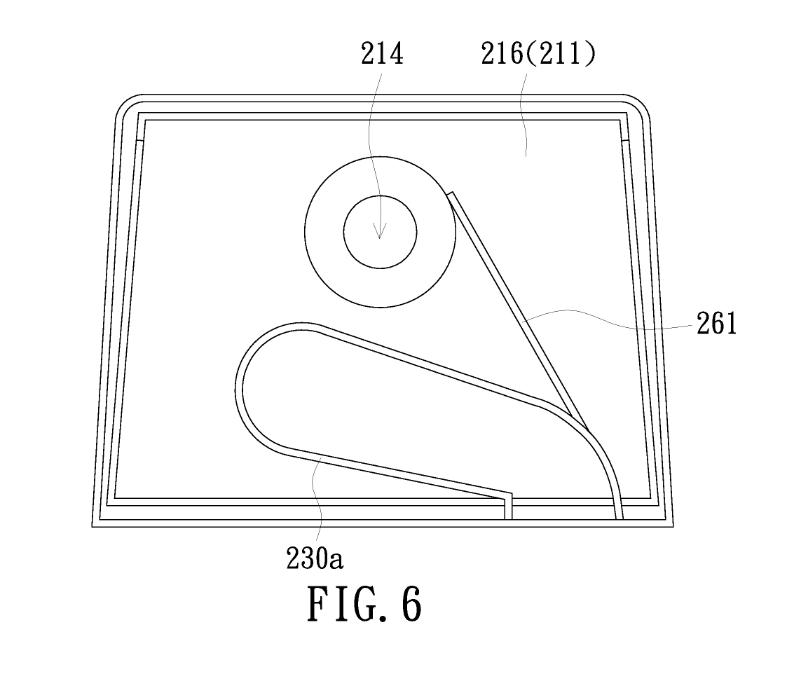

FIG. 6 is a schematic structural view of the cover body and the airway structure in FIG. 3 after being assembled. In this embodiment, as shown in FIG. 6, the top 211 (that is, the cover body 216) of the water container 210a may further include a dividing portion 261, extending toward the internal of the water container 210a, to divide the internal space of the water container 210a into a plurality of relatively small spaces; thus, the noise generated during the air humidification process is further reduced. Additionally, in another embodiment, the number of the dividing portion 261 may be more than one.

It is to be noted that the water container 210 in the humidifier 200 of FIG. 2 may be replaced by the water container 210a in the humidifier 200a of FIG. 3. Or, the water container 210a in the humidifier 200a of FIG. 3 may be replaced by the water container 210 in the humidifier 200 of FIG. 2.

In summary, because the airway structure is not in contact with the top of the water container and the internal space of the water container is divided into a plurality of relatively small spaces by the dividing plate, the noise generated during the air humidification process of the humidifier of the present invention is reduced. Accordingly, the breathing apparatus equipped with the humidifier of the present invention has reduced noises and consequentially a user can have good sleep quality.

The foregoing description of the preferred embodiment of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like is not necessary limited the claim scope to a specific embodiment, and the reference to particularly preferred exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the invention as defined by the following claims. Moreover, no element and component in the disclosure is intended to be dedicated to the public regardless of whether the element or component is explicitly recited in the following claims. Furthermore, the terms such as the first stop part, the second stop part, the first ring part and the second ring part are only used for distinguishing various elements and do not limit the number of the elements.

What is claimed is:

1. A humidifier, comprising:
a water container, having a top and a bottom opposite to the top, wherein the top of the water container has a first air outlet;
a dividing plate, disposed in the water container and dividing an internal space of the water container into a first space and a second space, wherein the first space is defined between the bottom of the water container and the dividing plate and the second space is defined between the dividing plate and the top of the water container; and
an airway structure, disposed in the second space and having a certain distance to the top of the water container, wherein the airway structure has an air inlet and a second air outlet, the air inlet of the airway structure is connected to a sidewall of the water container and the second air outlet of the airway structure is connected to the dividing plate.

2. The humidifier according to claim 1, wherein the airway structure comprises:
a fence, disposed on the dividing plate; and
a cover plate, disposed above the fence and having a certain distance to the top of the water container, and an airway space is formed by the cover plate, the fence and the dividing plate.

3. The humidifier according to claim 2, further comprising a connection structure connected between the cover plate and the top of the water container.

4. The humidifier according to claim 3, wherein the connection structure and the cover plate are integrated to have a one-piece structure.

5. The humidifier according to claim 2, wherein the fence and the dividing plate are integrated to have a one-piece structure.

6. The humidifier according to claim 1, wherein the airway structure is an airway pipe.

7. The humidifier according to claim 1, wherein there is a certain distance between the dividing plate and the sidewall of the water container.

8. The humidifier according to claim 1, wherein the dividing plate has at least one through hole.

9. The humidifier according to claim 1, wherein the top of the water container further has a dividing portion extending toward the internal of the water container.

10. The humidifier according to claim 1, wherein the air inlet is formed close to the top of the water container.

11. The humidifier according to claim 1, wherein the water container comprises:
a box body, having a top opening and a bottom opening opposite to the top opening;
a cover body, disposed on the top opening to form the top of the water container; and
a metal bottom plate, disposed on the bottom opening to form the bottom of the water container.

12. A breathing apparatus, comprising:
a humidifier, comprising:
a water container, having a top and a bottom opposite to the top, wherein the top of the water container has a first air outlet;
a dividing plate, disposed in the water container and dividing an internal space of the water container into a first space and a second space, wherein the first space is defined between the bottom of the water container and the dividing plate and the second space is defined between the dividing plate and the top of the water container; and
an airway structure, disposed in the second space and having a certain distance to the top of the water container, wherein the airway structure has an air inlet and a second air outlet, the air inlet of the airway structure is connected to a sidewall of the water container and the second air outlet of the airway structure is connected to the dividing plate;
a heating device, for supporting the humidifier and heating the bottom of the water container; and
an air supply device, for providing an air, wherein the air flows into the humidifier through the air inlet of the humidifier.
